(19) United States
(12) Patent Application Publication
(10) Pub. No.: US 2009/0101506 A1
(43) Pub. Date: Apr. 23, 2009

(54) METHOD, SPECIFIC PRIMERS AND KIT FOR THE DETECTION OF NEISSERIA MENINGITIDIS

(76) Inventor: Ivano Raffaele Victorio de Filippis Capasso, Rio de Janeiro (BR)

Correspondence Address: KLARQUIST SPARKMAN, LLP, 121 SW SALMON STREET, SUITE 1600, PORTLAND, OR, 97204, US

(21) Appl. No.: 11/597,531

(22) PCT Filed: May 24, 2005
(86) PCT No.: PCT/BR2005/000091

§ 371 Date: Jan. 28, 2008

(30) Foreign Application Priority Data
May 24, 2004 (BR) PI 0401929

(51) Int. Cl. G01N 27/447; C07H 21/04; C12Q 1/68

(52) U.S. Cl. 204/461; 536/24.33; 435/6

(57) ABSTRACT

The current invention refers to a Neisseria meningitidis detection method that is rapid, cheaper, more sensible, and more specific that those traditionally used for the same purpose. The invention is also related to specific primers to be used in the detection of Neisseria meningitidis by the Polymerase Chain Reaction, and includes a kit that contains such primers as its main elements.

The current invention refers to a method to detect Neisseria Meningitidis as well as specific primers to be used in this detection by the Polymerase Chain Reaction and a kit containing such primers as its main elements.

### FUNDAMENTALS OF THE INVENTION

The Meningococcal Meningitis is a serious disease, whose etiologic causing agent is the bacterium Neisseria meningitidis. The symptoms of this disease evolve rapidly, and can cause permanent damage to the patient's central nervous system, or even decease in few hours. Therefore, the Meningococcal Meningitis, when not diagnosed and treated on time, can be lethal in up to 30% of confirmed cases.

Laboratory exams to diagnose this disease are based on the research and detection of this bacterium in the clinical material, where it is found during the infection process, that is, in the blood fluid. However, after the collection of the clinical material, the high sensibility of the microorganism to external agents and to previous antibiotic treatments in patients with suspected meningococcal meningitis, make the cultivation of this agent difficult, which, in 96% of the cases, lead to false negative results.

Another diagnostic form for this disease is through direct serology of the clinical material from the patient. Yet, this method is also affected by the intensity grade of Neisseria meningitidis when outside the host body. This method also has the disadvantage of posing the possibility of cross-reaction occurrences with other microorganisms, which leads to false positive results.

Subsequently, in the last years, many studies have been conducted in such way to overcome the previously mentioned obstacles. Some employ, as a target, different regions of the Neisseria meningitidis genome, such as the genes rRNA 16S-23S (L. M. C., Hall, B., Duke, G., Urwin, An approach to the identification of the pathogens of bacterial meningitis by the polymerase chain reaction. Eur. J. Clin. Microbiol. Infect. Dis. 14:1090-1094, 1995. J. Xu, J. E., Millar, J. E., Moore, K., Murphy, H., Webb, A. J., Fox, M., Cafferkey, M. J., Crowe. Employment of broad-range 16S rRNA PCR to detect aetiological agents with acute meningitis—rapid separation of 16S rRNA PCR amplicons without the need for cloning. J. Appl. Microbiol. 94: 197-206, 2003. J. J., Lu, C. L., Perng, S. Y., Lee, C. C., Wan. Use of PCR with universal primers and restriction endonuclease digestions for detection and identification of common bacterial pathogens in cerebrospinal fluid. J. Clin. Microbiol. 38 (6):2076-2080, 2000. J. H., Atobe, M. H., Hirata, S., Hoshino-Shimizu, M. Schmal, E. M., Mamikuza. One-step heminested PCR for amplification of Neisseria meningitidis DNA in cerebrospinal fluid. J. Clin. Lab. Anal 14: 193-199, 2000), others use specific genes or sequences such as the gene porA (M. A., Diggle, S. C., Clark, Detection and genotyping of menigococci using a nested PCR approach. J. Med. Microbiol. 52: 51-57, 2003), or a insertion sequence IS1106 (J., Newcombe, K., Cartwright, W. H., Palmer, J., Mc Dfadden. PCR of peripheral blood for diagnosis of meningococcal disease. J. Clin. Microbiol. 34(7): 1637-1640, 1996. H., Ni, A. I., Knight, K., Cartwright, W. H., Palmer, J., Mc Fadden. Polymerase Chain Reaction for diagnosis of meningococcal meningitis. The Lancet 340: 1432-1434, 1992. R., Borrow, M., Guiver, F., Sadler, E. B., Kaczmarski, A. J., Fox. False positive diagnosis of meningococcal infection by the IS1106 PCR Elisa. FEMS Microbiol. Lett. 162: 215-218, 1998), or the gene ctrA (N., Lansac, F. J., Picard, C., Menard, M., Boissinot, M., Ouellette, P. H., Roy, M. G., Bergeron. Novel genus-specific PCR-based assays for rapid identification of Neisseria species and Neisseria meningitis. Eur. J. Clin. Microbiol. Infect. Dis. 19(6): 443-51, 2000), or the gene dhps (B. E., Kristiansen, E., Ask., A., Jenkis, C., Fermer, P., Radstrom, O., Skold. Rapid diagnosis of meningococcal meningitis by Polymerase Chain Reaction. The Lancet 337: 1568-1569, 1991), or automated PCR based on methods for laboratory confirmation of Meningococcal Meningitis (M. A., Diggle, G. P. S., Edwards, S. C., Clarke. Automation of fluorescence-based PCR for confirmation of meningococcal disease. J. Clin. Microbiol. 39(12): 4518-4519, 2001. M., Guiver, R., Borrow, J., Marsh, S. J., Gray, E. B., Kaczmarski, D., Howells, P., Boseley, A. J., Fox. Evaluation of the applied biosystems automated Taqman polymerase chain reaction system for the detection of meningococcal disease. FEMS Imm. Mol. Microbiol. 28: 173-179, 2000. M. A., Diggle, S. C., Clark. Semi-automation of the polymerase chain reaction for laboratory confirmation of meningococcal disease. Br. J. Biochem. Sci. 59(3): 137-140, 2002).

However, some of the previously cited methods are not sufficiently sensitive and specific for the detection of Neisseria meningitidis directly from clinical samples. (D. N., Fredricks, D. A., Relman. Application of polymerase chain reaction to the diagnosis of infectious diseases. Clin. Inf. Dis. 29: 475-88, 1999. J. H., Atobe, M. H., Hirata, S., Hoshino-Shimizu, M. Schmal, E. M., Mamizuka. One-step heminested PCR for amplification of Neisseria meningitidis DNA in cerebrospinal fluid. J. Clin. Lab. Anal 14: 193-199, 2000. R., Borrow, M., Guiver, F., Sadler, E. B., Kaczmarski, A. J., Fox. False positive diagnosis of meningococcal infection by the IS1106 PCR Elisa. FEMS Microbiol. Lett. 162: 215-218, 1998. N., Lansac, F. J., Picard, C., Menard, M., Boissinot, M., Ouellette, P. H., Roy, M. G., Bergeron. Novel genus-specific PCR-based assays for rapid identification of Neisseria species and Neisseria meningitides. Eur. J. Clin. Microbiol. Infect. Dis. 19(6): 443-51, 2000), while others are slow, requiring a Polymerase Chain Reaction in two stages (L. M. C., Hall, B., Duke, G., Urwin, An approach to the identification of the pathogens of bacterial meningitis by the polymerase chain reaction. Eur. J. Clin. Microbiol. Infect. Dis. 14:1090-1094, 1995. J. Xu, J. E., Millar, J. E., Moore, K., Murphy, H., Webb, A. J., Fox, M., Cafferkey, M. J., Crowe. Employment of broad-range 16S rRNA PCR to detect aetiological agents of infection from clinical speciments in patients with acute meningitis—rapid separation of 16S rRNA PCR amplicons without the need for cloning. J. Appl. Microbiol. 94: 197-206, 2003. J. J., Lu, C. L., Perng, S. Y., Lee, C. C., Wan. Use of PCR with universal primers and restriction endonuclease digestions for detection and identification of common bacterial pathogens in cerebrospinal fluid. J. Clin. Microbiol. 38(6):2076-2080, 2000) or the re-amplification of a PCR product, establishing a denominated Nested-PCR (D. N., Fredricks, D. A., Relman. Application of polymerase chain reaction to the diagnosis of infectious diseases. Clin. Inf. Dis. 29: 475-88, 1999. M. A., Diggle, S. C., Clark. Detection and genotyping of meningococci using a nested PCR approach. J. Med. Microbiol. 52: 51-57, 2003). Other method pose the disadvantage of requiring the use of costly reagent equipment, which are not feasible for the majority of developing countries (M. A., Diggle, G. F. S., Edwards, S. C., Clarke. Automation of fluorescence-based PCR for confirmation of meningococcal disease. J. Clin. Microbiol. 39(12): 4518-4519, 2001. M., Guiver, R., Borrow, J., Marsh, S. J., Gray, E. B., Kaczmarski, D., Howells, P., Boseley, A. J., Fox. Evaluation of the applied biosystems automated Taqman polymerase chain reaction system for the detection of meningococcal disease. FEMS Imm. Mol. Microbiol. 28: 173-179, 2000. M. A., Diggle, S. C., Clark. Semi-automation of the polymerase chain reaction for laboratory confirmation of meningococcal disease. Br. J. Biochem. Sci. 59(3): 137-140, 2002).

Hence, it becomes necessary to develop a diagnostic methods for Neisseria meningitis, which dos not pose the previously mentioned disadvantages.

### SUMMARY OF THE INVENTION

The main objective of this invention is to detect the Neisseria meningitidis through a method that is quick, cheaper, more sensible and more specific than those traditionally employed for the same purpose.

A first concretization of this invention refers to the specific primer for the detection of the Neisseria meningitidis, which shows a sequence as described in SEQ. ID No. 1 or in sequences with equivalent functionalities.

A second concretization of this invention refers to the specific primer for the detection of the Neisseria meningitis, which shows a sequence as described in SEQ. ID No. 2 or in sequences with equivalent functionalities.

A third concretization of this invention refers to the specific primer for the detection of the Neisseria meningitidis, where the amplification and identification of the entire or part of a sequence of nucleotides, which are a characteristic of the Neisseria meningitidis, as described in SEQ. ID No. 5 or in sequences with equivalent functionalities. (This referred method employs the specific primers as described in SEQ. ID No. 1 and SEQ. ID No. 2, or in sequences with equivalent functionalities, and is characterized by the following phases:

(a) collect the sample to be examined;

(b) extract the sequence of nucleotides from the sample obtained in phase (a);

(c) amplify the sequence of nucleotides obtained in phase (b) through the polymerase chain reaction using, as primers, those that correspond to SEQ. ID No. 1 and SEQ ID No. 2 or in sequences with equivalent functionalities;

(d) separate the products amplified in phase (c) by electrophoresis, followed by the detection using an appropriate technique.

A fourth concretization of this invention is related to a primer for the confirmation of dubious samples after phase (d) of the above mentioned method, which shows the sequence as described in SEQ. ID No. 3, or in sequences with equivalent functionalities.

A fifth concretization of this invention is related to another primer for the confirmation of dubious samples after phase (d) of the above mentioned method, which shows the sequence as described in SEQ. ID No. 4, or in sequences with equivalent functionalities.

A sixth concretization of this invention refers to a kit used for the amplification identification of the entire or a part of the sequence of nucleotides, which is an exclusive characteristic of the Neisseria meningitidis (as described in SEQ. ID No. 5 or in sequences with equivalent functionalities), which contains, as main elements, specific primers as described in SEQ. ID No. 1 and SEQ. ID No. 2 (or in sequences with equivalent functionalities). A basic kit comprehends, in addition to the above-mentioned primers, the primers that correspond to SEQ. ID No. 3 and SEQ. ID No. 4 or sequences with equivalent functionalities when there is the need to confirm doubtful samples and all the necessary agents in order to employ the Polymease Chain Reaction (PCR) technique, namely: nucleotides and an appropriate buffer solution for the amplification by PCR. Alternatively, the kit may contain a sufficient quantity of enzyme Taq polymerase for amplification, standard DNA to be used as a positive control of the reaction, a buffer solution of the sample to prepare the amplified material for electrophoresis, and protocol and manual to instruct the user.

### BRIEF DESCRIPTION OF THE DESIGNS

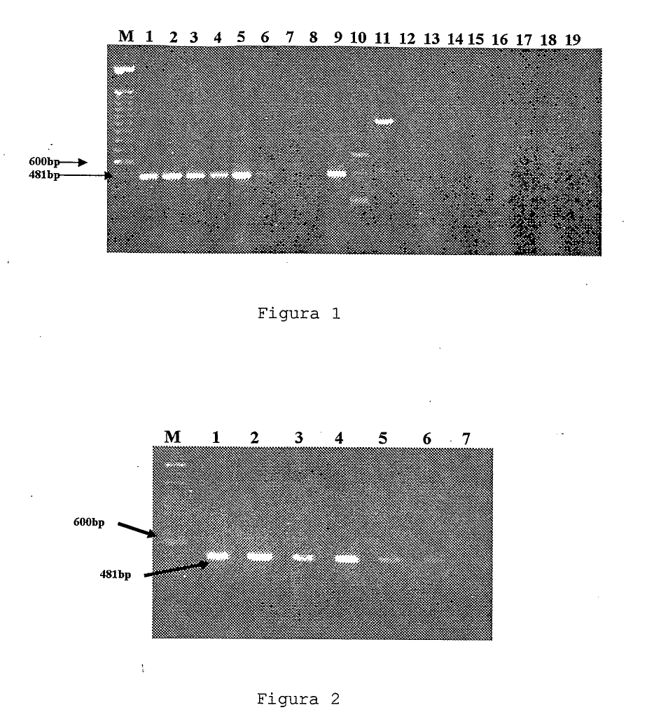

FIG. 1: Polymerase Chain Reaction (PCR) of different bacterial species to determine method specifications.

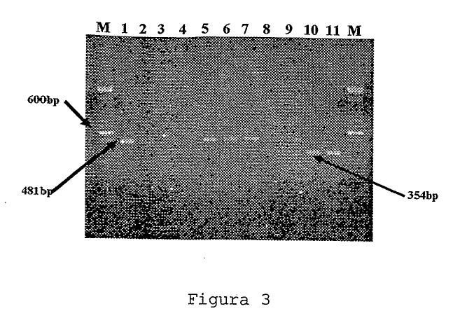

FIG. 2: Methods sensibility: serial dilution of genomic DNA of Neisseria meningitidis, analyzed by the described method of electrophoresis in agarose gel. The arrow indicates the expected size of the band, indicating a positive reaction (481b).

FIG. 3: “Nested-PCR” of Cerebrospinal Fluid samples.

### DETAILED DESCRIPTION OF THE INVENTION

Using this invention, the bacterium Neisseria meningitidis is detected through a method that is quicker, cheaper, more sensitive, and more specific than those traditionally employed for the same purpose. This method, which specifically detects the entire or a part of a characteristic sequence of Neisseria meningitidis, as described in SEQ ID No. 5 or in sequences with equivalent functionalities, includes the following phases:

(a) collect the sample to be examined;

(b) extract the bacterial genomic DNA containing the sequence of nucleotides from the sample obtained in phase (a);

(c) amplify the sequence of nucleotides obtained in phase (b) through the polymerase chain reaction, using those primers that correspond to SEQ. ID No 1 and SEQ. ID No. 2 or sequences with equivalent functionalities;

(d) separate the products amplified in phase (c) by electrophoresis, followed by the detection, using an appropriate technique.

The above-mentioned primers were designed during the research that originated the current invention, and includes the sequences described in SEQ. ID No. 1 and SEQ. ID No. 2 or in sequences of equivalent functionalities.

The sequences are denominated equivalent if the correspondent biopolymer functionalities can play the same role, even if not identical, of the considered utilization or application. The equivalent sequences can be a result from the variability, that is, any spontaneous or induced modification of a sequence, from the substitution and/or deletion and/or insertion of nucleotides, and/or extension and/or shortening of the sequence at one of the extremes. A no-natural variability can result from genetic engineering techniques.

Therefore, as previously described, when this couple of primers, or their equivalent functionalities, are used in the Polymerase Chain Reaction, they afford the detection of the Neisseria meningitidis DNA directly from the clinical material collected from the patient, even if it is not possible to cultivate the microorganism by traditional methods.

Using the method of this invention, results are obtained in 3 hours approximately. In addition, this method can afford a 100% sensibility and specification, other than not being expensive in comparison to other existing state-of-art methods used for the same purpose.

In case of dubious results after the detection in phase (d) of the method of this invention, a second amplification phase can be made, which employs the primers correspondent to SEQ. ID No. 3 and SEQ. ID No. 4, or sequences with equivalent functionalities.

Furthermore, this invention also encloses a kit for the amplification and identification of the entire or part of the sequence of nucleotides, which are a characteristic of Neisseria meningitidis (as described in SEQ. ID No. 5 or in sequences with equivalent functionalities). This kit contains such primers as main elements. Furthermore, the kit comprehends, in addition to the above-mentioned primers, the primers correspondent to SEQ. ID. No. 3 and SEQ. ID No. 4, or sequences with equivalent functionalities when there is the need to confirm dubious samples and all the necessary agents in order to employ the Polymease Chain Reaction (PCR) technique, namely: nucleotides and an appropriate buffer solution for the amplification through PCR. Alternatively, the kit may contain a sufficient quantity of enzyme Taq polymerase for the amplification, standard DNA to be used as a positive control of the reaction, a buffer solution of the sample to prepare the amplified material for electrophoresis, and protocol and manual to instruct the user.

In this invention, the detection of Neisseria meningitidis genomic DNA is preferably taken from the Cerebrospinal Fluid (CSF). The DNA detection from blood or serum requires a greater number of phases, or the utilization of more expensive kits, as compared to the extraction of DNA from CSF. Moreover, the symptoms of meningococcal meningitis become clearer only during the meninx infection, which characterizes the CSF as the preferred material for the detection of this bacterium's DNA.

In addition, the products for amplification of phase (c), of the method of this invention, can be separated by techniques known by experts, such as electrophoresis in agarose gel, and observing the band after being colored by ethidium bromide.

This invention is described in details through the examples illustrated below. It is necessary to emphasize that the invention is limited to these examples, but also include variations and modifications within it functional limits.

### EXAMPLE 1

### Design of the Primers

The primers of this invention were designed from the sequence of the gene NspA of Neisseria meningitidis, which is available at the GeneBank under the number AF175683, whose partial sequence is describe in the SEQ ID No 5. For the referred design, the program Primer 3: WWW Primer Tool (Whitehead Institute for Biomedical Research) was used.

The Gene NspA is a region of 525 pb, which is present in Neisseria meningitidis and Neisseria gonorrheae genomes, codifying for the synthesis of a protein from a conserved external membrane. However, although it is also present in the Neisseria gonorrheae genome, its utilization to mold the construction of primers for the invention was not invalidated once there are no reports of the Neisseria gonorrheae genome in materials utilized for the detection of Neisseria meningitidis.

The sequence of the obtained indicators are illustrated in Table 1.

TABLE 1

Specific Neisseria meningitidis Primers.

| | |
|---|---|
| Primer | Sequence |

| | |
|---|---|
| NspA1 | AGCACTTGCCACACTGATTG |
| (SEQ. ID. No. 1) | |
| NspA-2 | GGAACGGACGTTTTTGACAG |
| (SEQ. ID. No. 2) | |

### EXAMPLE 2

### Materials and Methods Used

### A) Patients:

A hundred and twelve patients showing meningococcal Meningitis symptoms (high fever, vomit, cutaneous eruptions, strong headaches, neck stiffness) were enrolled at the Instituto Estadual de Infectologia de São Sebastião (IEISS) [State Infectology Institute of São Sebastião] during the 1999-2000 period. Immediately after the enrollment, all patients were submitted to a femoral puncture to collect cerebrospinal fluid (CSF).

Nearly 0.3 to 1.0 ml of CSF were collected. The exceeding CSF not used was frozen at −25° C. [−13° F.], and analyzed subsequently (around two or three days after).

### B) CSF Strains and Samples:

A total of 83 Neisseria meningitidis strains were tested, including the serum groups A, A, C, and W135, which were isolated from the CSF from patients with meningococcal meningitis who were admitted in public hospitals in different Sates through the period between 1990 and 2002. A hundred and twelve samples of CSF collected from patients hospitalized because of suspected meningococcal meningitis in Rio de Janeiro between 1999 and 2000 were included in the current study. There were also 5 strain of Neisseria meningitidis tested (from subgroups A, B, C, and W135), 4 strains referred to Neisseria s, and 8 strains of referred to other bacterial species which were frequently associated with meningitis. These accounted for a total of 100 tested strains. Table 2 shows the referred strains.

TABLE 2
Bacterial species utilized to test the method
specification; (MD), Meningococcal Disease.

| Species | Origin | No of strains | No positives |
|---|---|---|---|
| N. meningitidis sA | MD, Brazil | 4 | 4 |
| N. meningitidis sB | MD, Brazil | 57 | 57 |
| N. meningitidis sC | MD, Brazil | 16 | 16 |
| N. meningitidis sW135 | MD, Brazil | 6 | 6 |
| N. meningitidis sA | ATCC 13077 | 1 | 1 |
| N. meningitidis sB | ATCC 13090 | 1 | 1 |
| N. meningitidis sC | ATCC 13102 | 1 | 1 |
| N. meningitidis sD | ATCC 13313 | 1 | 1 |
| N. meningitidis sW135 | ATCC 35559 | 1 | 1 |
| N. gonorrhoeae | ATCC 19424 | 1 | 1 |
| N. gonorrhoeae | ATCC 9826 | 1 | 1 |
| N. lactamica | ATCC 23970 | 1 | 0 |
| N. subflava | ATCC 11076 | 1 | 0 |
| H. influenzae stype b | ATCC 10211 | 1 | 0 |
| H. influenzae stype b | ATCC 33533 | 1 | 0 |
| S. pneumoniae | ATCC 33400 | 1 | 0 |
| S. agalactiae | ATCC 13813 | 1 | 0 |
| K. pneumoniae | ATCC 13883 | 1 | 0 |
| L. monocytogenes | ATCC 15313 | 1 | 0 |
| Acinetobacter sp. | ATCC 14293 | 1 | 0 |
| E. coli | ATCC 11775 | 1 | 0 |

In addition, a total of 112 CSF samples (only CSF, and not isolated strains) were also tested, being collected between 1999 and 2000 from patient with meningococcal Disease (MS), who hospitalized in public hospitals of Rio de Janeiro.

### C) DNA Extraction and Quantification

To employ the Polymerase Chain Reaction (PCR), two extraction methods were tested:

a) Brutal Extraction:
CSF: the laboratory received around 50 μl to 500 ul of Cerebrospinal Fluid.
Strains: Neisseria meningitidis cells were obtained from the “Agar Chocolate” (Blood with agar basis, with 5% of blood from a sterile defibrinated rabbit, and induced to thermal shock lysate when added to the basis) and ressuspended in 400 μl of ultra-pure water for PCR.
DNA Extraction: the CSF and the bacterial suspension in water were heated at 95° C. [203° F.] for 10 minutes, and centrifuged at 13000×g per minutes. 2.5 μl of the supernatant was employed as a mold for PCR, and discharged afterwards.
b) Purified Extraction: the purified DNA of CSF and from the referred strains were obtained with Qiagen “Tissue Kit” (Qiagen GmbH, Hilden, Germany) following indications from the manufacturer.

### D) Conventional Laboratory Tests:

a) Oxidize Culture and Test:

From 0.2 to 0.5 ml of CSF samples from patients clinically diagnosed with meningococcal meningitis were used in the test. These were inoculated in agar chocolate plaques with a “Mueller-Hinton” base, having 10% of blood from a sterile defibrinated sheep and induced to thermal shock lysate when added to the base. The agar plaques were placed in a microaerophilia jar with 5% of CO₂ incubated at 37° C. [99° F.] during 48 hours. After this period, the brown-colored colonies were collected from the chocolate agar and tested against the oxidize production. For this test, a colony was collected with a bacteriological loop, and a smear was applied to this colony on a piece of saturated paper with N′-N′-tetrametil-p-phenylene diamine. The purple color turn-out in the smear place was considered a positive reaction. The Gram color of the collected colonies from the agar chocolate was also employed. The presence of gram negative diplococcus, detected by microscopy, a positive oxidize test, and the presence of brow-colored colonies in the agar chocolate AC plaque after 48 hours of incubation in a microaerophilia jar, confirm the presence of Neisseria meningitidis in the inoculated CSF.

b) Agglutination by Latex:

One drop of CSF was ressuspended in a drop of latex antiserum (Bio Mérieux—Slidex Méningite—Kit 5) for the serum-groups A, B, C, and latex antiserum (DIFCO—Neisseria meningitidis Antiserum) for the serum-groups D, W135, Y, and Z. A visible agglutination after 30-60 seconds was considered positive for the specifically tested serum-group.

### EXAMPLE 3

### Polymerase Chain Reaction

### NspA-PCR

The two extraction methods showed the same results with sufficient quantities of intact DNA for PCR. The brutal lysis method was the most rapid and simple, and the lysates could be used after the extraction in order to provide good-quality DNA for the PCR test. After the first PCR, the lysates were frozen at 20° C. [68° F.] and reused in the PCR in order to determine whether it was possible to conserve lysates for a second test, in case of necessity. However, the positive samples of the first PCR showed negative results after being frozen and unfrozen. Probably, it was due to nuclease actions that downgraded the genomic DNA other than having the presence of a single copy of the gene NspA in the Neisseria meningitidis genome. The Qiagen kit provided a highly purified DNA which could be used several times after frosting. The frequent frosting and defrosting procedures did not affect NspA-PCR results. Hence, one of these DNA samples was selected as positive controls in all PCR experiments, being employed for almost an year since the current invention without alterations in the PCR standard. Nevertheless, for the objectives of this invention, a quicker extraction method was selected (brutal lysis) in order to demonstrate how NspA-PCR can confer a definite result within only few hours.

PCR was employed in a single tube (in a single stage) with a reaction volume of 25 μl containing 1×PCR Buffer (Invitrogen Co. Carlsbad, Calif., USA), 2 mM MgCl₂, 6% DMSO, 200 μM of each on of dATP, dCTP, dGTP and dTTP (Invitrogen Co. Carlsbad, Calif., USA), 50 μmol of each primer, 1 U of polymerase Taq (Invitrogen Co. Carlsbad, Calif., USA) and 2.5 μl of DNA mold. The PCR cycles were done in a MJ PT-200 thermocycler with the following parameters: 5 min. at 94° C. [201° F.] 1 min. at 94° C. [201° F.], 1 min. at 60° C. [140° F.] and 2 min. at 72° C. [162° F.], with a final extension of 7 min. at 72° C. [162° F.].

### EXAMPLE 4

### Analysis on the Polymerase Chain Reaction (PCR) Products

A sample of 10 μl of PCR products was applied in agarose gel and submitted to electrophoresis at 100V for 30 minutes in 0.5× Tri-borate-EDTA buffer (pH=8.0), having a molecular weight marker of 100 pb (Invitrogen Co. Carlsbad, Calif., USA). The gels were colored with ethidium bromide and the images were digitized through Video Documentation System, and analyzed with the program ImageMaster (Amersham Pharmacia Biotech).

The quantity of purified DNA after the extraction was determined by a digital spectrophotometer at 0.260 nm and diluted at 10⁻⁶ in serial dilutions of 1:10. The Neisseria meningitidis DNA detection limit was determined using such dilutions as molds for the NspA-PCR (FIG. 2).

All the Neisseria meningitidis strains isolated from the PCR produced, after the PCR with NspA-1 and NspA-2 primers, a band of 481 pb, including the strains referred to Neisseria meningitidis and Neisseria gonorrheae. FIG. 1 illustrates such results.

In FIG. 1, the line M corresponds to the molecular weight marker (Invitrogen Co. Carlsbad, Calif., USA). Line 1, 2, 3, and 4 correspond, respectively, to results referred to Neisseria meningitidis A (ATCC 13077), Neisseria meningitidis B (ATCC 13077), Neisseria meningitidis C (ATCC 13102), and D (ATCC 13313). As to lines 5, 6, 7, 8, 9, and 10, these correspond respectively, to results associated to Neisseria meningitidis W135 (ATCC 35559), Neisseria gonorrhoeae (ATCC 19424), S. Pneumoniae (ATCC 33400), E. Coli (ATCC 11775), a Neisseria gonorrhoeae (ATCC 9826) and N. Lactamica (ATCC 23970). The lines 11, 12, 13, 14, 15, 16, 17, 18 and 19 are linked, respectively, to results of N. subflava (ATCC 11076), H. influenzae b (ATCC 10211), H. influenzae b (ATCC 33533), S. agalactiae (ATCC 13813), positive CSF, K. pneumoniae (ATCC 13883), L. monocytogenes (ATCC 15313), Acinetobacter sp. (ATCC 14293) and negative control.

The above-mentioned primers were designed to amplify the internal region of the gene NspA in order to prevent unspecific bond due to different non-codification region, on gene flank. Thus, no DNA from another Neisseria spp. and from other bacterial agents usually found in bacterial meningitis (except N. Lactamica, N. Subflava and L. monocytogenes) was amplified.

The N. Lactamica, N. Subflava and L. monocytogenes species showed unspecific amplification, having bands with molecular weights differently than expected. The NspA-PCR of N. Lactamica showed three bands, one of which had apparently the same molecular weight than the amplicons obtained from N. Meningitidis. However, the presence of other bands of approximately 700 and 300 pb, different from the 481 pb band which is common to all tested N. Meningitidis, was sufficient to discharge this strain as non-N. Meningitidis. N. Subflava and L. monocytogenes also showed unspecific bands with molecular weights differently than what is expected for N. Meningitidis. These results were also presented in FIG. 1.

The amplification of gene NspA was observed with a dilution of 10⁻⁶, corresponding to 267 fg of DNA, which corresponds to almost 90 copies of the genome. This result can be observed in FIG. 2. According to other authors, the methods sensibility based on PCR for N. Meningitidis DNA detection in clinic samples varies from 2 fg to 5 pg (Kotilainen, P., J. Jalava, O. Meurman, O. P. Lehtonen, E. Rintala, O. P. Seppala, E. Eerola and S. Nikkari. 1998. Diagnosis of meningococcal meningitis by broad-range bacterial PCR with cerebrospinal fluid. J. Clin. Microbiol. 36(8):2205-2209. Lansac, N., F. J. Picard, C. Menard, M. Boissinot, M. Ouellette, P. H. Roy and M. G. Bergeron. 2000. Novel genus-specific PCR-based assays for rapid identification of Neisseria species and Neisseria meningitidis. Eur. J. Clin. Microbiol. Infect. Dis. 19(6):443-51. Martin, D., N. Cadieux, J. Hamel and B. R. Brodeur. 1997. Highly Conserved Neisseria meningitidis Surface Protein Confers Protection against Experimental Infection. J. Exp. Med. 185(7):1173-1183. Ni, H., A. I. Knight, K. Cartwright, W. H. Palmer and J. Mc Fadden. 1992. Polymerase Chain Reaction for Diagnosis of Meningococcal Meningitis. The Lancet 340:1432-1434).

FIG. 2 corresponds to the sensibility NspA-PCR. The dilution series of N. meningitidis were analyzed by NspA-PCR and electrophoresis in gel, corresponding to 26.7 ng (line 1), 2.67 ng (line 2), 267 pg (line 3), 26.7 pg (line 4), 2.67 (line 5) and 267 pg (line 6). Line M is the molecular weight marker of 100 pb. the arrow indicates the specific band size (481 pb).

When comparing the etiologic agent detection to the menigococcal meningitis by conventional laboratory methods (observed by direct microscopy, culture and serology of agglutination by latex) and by NspA-PCR from the CSF samples, the result of this invention show that 21 of the 112 (18.75%) negative samples resulted by conventional method, and of clinically suspected cases of meningococcal meningitis were positives when employed by NspA-PCR. In addition, all CSF positive samples obtained through any conventional laboratory method (n=51), were also positive through NspA-PCR. Table 3 shows these data.

TABLE 3
Comparison between convetional disgnostic methods
(culture, direct microscopy, and serology) and NspA-PCR.

| Conventional Methods | NspA-PCR | |
|---|---|---|
| (CSF) | (positives/total) | Sensibility |
| Positive Serology | 44/112 (39.2%) | 100% |
| (n = 44) | | |
| Positive Culture | 37/112 (33%) | 100% |
| (n = 37) | | |
| Positive Microscopy | 22/112 (19.6%) | 100% |
| (n = 22) | | |
| Any positive method | 51/112 (45.5%) | 100% |
| (n = 51) | | |
| All negative methods | 0/112 (0%) | NA^{a} |
| (n = 32) | | |

Therefore, for NspA-PCR, sensibility and specification were, respectively, 100% and 78.4%.

Additionally, clinical data of patients from public hospitals with meningococcal meningitis symptoms showed a low number of patients, who were submitted to antibiotic therapy before being hospitalized (5 of 112, or 4.5%), and of CSF collection for laboratory analysis. All 5 patients showed clear meningococcal meningitis symptoms, and 2 of these also had a positive diagnosis in convention laboratory tests. Furthermore, 3 out of 112 patients (2.7%) deceased while receiving hospital care. Two of these three casualties are associated to patients that did not show clear meningococcal meningitis symptoms and had negative results by conventional detection methods, but positive PCR. The other casualty happened within the group of patients without meningococcal meningitis symptoms, but having positive results in both conventional methods and PCR. Table 4 illustrates the referred data.

TABLE 4
Consistency between conventional laboratory
methods (LAB), meningococcal disease symptoms (MD) and PCR
of the CSF for the diagnosis on meningococcal meningitis in
112 infected patients.

| | PCR (+) | PCR (−) | PPV | NPV | TOTAL |
|---|---|---|---|---|---|
| MD (+) | 5 (2)a | 0 | — | — | 5 |
| LAB (+) | | | | | |
| MD (−) | 11 [2]b | 29 | — | — | 40 |
| LAB (−) | | | | | |
| MD (+) | 21 | 0 | — | — | 21 |
| LAB (−) | (3)a [1]b | | | | |
| MD (−) | 46 [1]b | 0 | — | — | 46 |
| LAB (+−) | | | | | |
| TOTAL | 83 (74%) | 29 (26%) | 86.7% | 100% | 112 |

( )a = number of submitted patients with previous antibiotic therapy.
[ ]b = number of casualties.

### EXAMPLE 5

### Sequencing of Products by Polymerase Chain Reaction (PCR)

The amplified products of 481 pb, generated from 5 positive sampled of different years, were purified with QIAquick Nucleotide Removal Kit (Qiagen, GmbH, Hilden, Germany) and sequenced in a ABI 377 Automatic Sequencer utilizing the BigDye-Terminator Cycle Sequencing Kit (Applied Biosystem, Foster City, Calif. USA). The employed sequencing cycle parameters were exactly as described by the protocol from the manufactured, and the primers NspA-1 and NspA-2 utilized in the sequencing reaction were the same employed for NspA-PCR. All the sequences were determined at least twice for each DNA tape.

The comparison of the gene NspA obtained from the N. meningitidis, revealed a similarity that varied from 98 to 99% after the analysis using the program “BLAST” (National Center of Biotechnology Information, Bethesda, USA). These sequences were compared against six sequences of gene NspA stocked in the GenBank (National Center for Biotechnology Information, Bethesda, USA), whose access numbers are: AF175681, AL162754, AF175677, AF175679, AF175676, AE002420 (Table 5). The obtained sequences were aligned with the program BioEdit (BioEdit Sequence Alignment Editor, North Carolina State University, USA), showing small punctual alterations, which are being studied to determine the diversity of gene NspA between N. meningitidis strains isolated in Brazil.

TABLE 5
Homology of amplified gene NspA in five strains of
N. meningitidis isolated from CSF, with 6 sequences of gene
NspA stocked in the GenBank.

| | | HOMOLOGY | | | | | | |
|---|---|---|---|---|---|---|---|---|
| Strain/year | | Serum | AF17568 | AL162754 | AF175677 | AF175679 | AF175676 | AE002420 |
| Isolation | Origin | group | NmNG3/88 | Nm Z2491 | Nm BZ232 | Nm M136 | Nm 8047 | Nm MC58 |
| P415/1991 | Brazil (SE) | B | 98% | 98% | 98% | 98% | 98% | 97% |
| P2149/1999 | Brazil (NE) | B | 99% | 99% | 99% | 99% | 99% | 98% |
| P2355/2001 | Brazil (S) | B | 99% | 99% | 99% | 99% | 99% | 98% |
| P2363/2000 | Brazil (S) | B | 99% | 99% | 99% | 99% | 99% | 99% |
| P2498/2002 | Brazil (SE) | C | 98% | 99% | 99% | 99% | 99% | 99% |

SE = southeast region,
NE = northeast region,
S = south region.

### EXAMPLE 6

### Nested Polymerase Chain Reaction

### Nested NspA-PCR

In order to confirm the negative samples after employing the PCR, a second amplification reaction was made using a new coupe of primers designed to amplify an internal regional of the first amplicon of 481 pb. Table 6 shows the used primers in the second amplification reaction.

TABLE 6

Primers employed in Nested -PCR

| | |
|---|---|
| Primer | Sequence |

| | |
|---|---|
| NspA Nest-1 | TAGGTTCTGCCAAAGGCTTC |
| (SEQ.ID. No. 3) | |
| NspA Nest 2 | CAGTGTTGACTTTGCCGATG |
| (SEQ.ID. No. 4) | |

For NspA-PCR products that show weak bands, this Nested NspA-PCR afforded the amplification of a fragment of a 354 pb DNA. This fragment was easily observed after the electrophoresis in colored gel with ethidium bromide, confirming the presence of the N. meningitidis DNA in the CSF. FIG. 3 illustrates such results.

In FIG. 3, the M line represents the molecular weight marker of 100 pb. Lines 1, 5, 6, and 7 correspond to positive samples. Lines 2, 3, and 4 are associated to questionable samples. Line 8 corresponds to negative control, while line 9 confirm the negative sample of line 3 after NspA-PCR. Lines 10, and 11 confirm the positive samples of line 2 and 4 after NspA-PCR. The arrows indicate the molecular weight of the expected products of 418 pb (NspA-PCR) and 354 pb (Nested NspA-PCR).

The confirmation of Nested NspA-PCR was employed with all samples with weak bands or with no bands after NspA-PCR. Seven out of 83 positive samples (8.4%) showed weak bands after NspA-PCR and were submitted to Nested NspA-PCR. All these seven samples showed a fragment of 354 pb DNA, which confirm the presence of N. meningitidis DNA in the CSF. In addition, 29 negative samples after NspA-PCR were also submitted to Nested-NspA-PCR, showing negative results with no DNA amplification.

Both NspA-PCR and Nested-NspA-PCR were employed several times in order to confirm the results that can be reproduced for the amplification of the gene NspA.

1. Oligonucleotide primer characterized by comprehending the sequence SEQ ID No. 1 or sequences with equivalent functionalities.

2. Oligonucleotide primer characterized by comprehending the sequence SEQ ID No. 2 or sequences with equivalent functionalities.

3. Oligonucleotide primer characterized by comprehending the sequence SEQ ID No. 3 or sequences with equivalent functionalities.

4. Oligonucleotide primer characterized by comprehending the sequence SEQ ID No. 4 or sequences with equivalent functionalities.

5. Detection method of nucleotide sequence, characteristic of Neisseria meningitidis, characterized by the fact that the sequence corresponds to the entire or part of the SEQ ID No. 5 or sequences with equivalent functionalities and containing the following phases:
(a) collect the sample to be examined;
(b) extract the sequence of nucleotides from the sample obtained in phase (a);
(c) amplify the sequence of nucleotides obtained in phase (b) through the polymerase chain reaction using, as primers, those that correspond to SEQ. ID No. 1 and SEQ ID No. 2 or sequences with equivalent functionalities;
(d) separate the products amplified in phase (c) by electrophoresis, followed by the detection using the appropriate technique.

6. Method according to claim 5 characterized by the fact the amplification products of phase (d) are separated by electrophoresis in agarose gel and detected by ethidium bromide coloration.

7. Method according to claim 5 characterized by the fact that amplification products of phase (c) are submitted to a second amplification phase in order to confirm dubious samples after the detection of phase (d).

8. Method according to claim 7 characterized by employing primers that correspond to SEQ ID No. 3 and SEQ ID No. 4 or sequences with equivalent functionalities in the second amplification phase.

9. Kit for amplification and identification of the entire or part of the nucleotide that are a characteristic of Neisseria meningitidis following the method described in claim 5 characterized by the fact of comprehending primers that correspond to SEQ ID No. 1 and SEQ ID No. 2 or sequences with equivalent functionalities, all necessary reagents and additives to employ the amplification by polymerase chain reaction, primers correspondent to SEQ ID No. 3 and SEQ ID No. 4 or sequences with equivalent functionalities incase of need to confirm dubious samples, and, alternatively, protocol and manual to instruct users.


SEQUENCE LISTING (5 sequences)

SEQ ID NO: 1 - DNA, 20 bp, Artificial Sequence
  Primer
agcacttgccacactgattg

SEQ ID NO: 2 - DNA, 20 bp, Artificial Sequence
  Primer
ggaacggacgtttttgacag

SEQ ID NO: 3 - DNA, 20 bp, Artificial Sequence
  Primer
taggttctgccaaaggcttc

SEQ ID NO: 4 - DNA, 20 bp, Artificial Sequence
  Primer
cagtgttgactttgccgatg

SEQ ID NO: 5 - DNA, 481 bp, Neisseria meningitidis
agcacttgccacactgattgccctcgctctcccggccgccgcactggcggaaggcgcatccggcttttacgtccaagccgatgccgcacacgcaaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgccatttacgacttcgacacccaatcgcccgtcaaaccgtatctcggcgcgcgcttgagcctcaaccgcgcctccgtcgacttgggcggcagcgacagcttcagccaaacctccatcggcctcggcgtattgacgggcgtaagctatgccgttaccccgaatgtcgatttggatgccggctaccgctacaactacatcggcaaagtcaacactgtcaaaaacgtccgttcc